SEQUENCE LISTING (35 sequences)

SEQ ID NO: 1 - DNA, 28 bp, Artificial
  synthetic PCR amplification Forward primer for beta3Gal-T5
tttgtcgacagtctaaatcctttcaaag

SEQ ID NO: 2 - DNA, 26 bp, Artificial
  synthetic PCR amplification Reverse primer for beta3Gal-T5
tttaagcttcagacaggcggacaatc

SEQ ID NO: 3 - DNA, 28 bp, Artificial
  synthetic PCR amplification Forward primer for beta3Gal-T1
tttggatccagtataactcgccctactt

SEQ ID NO: 4 - DNA, 25 bp, Artificial
  synthetic PCR amplification Reverse primer for beta3Gal-T1
tttaagcttcctaacatctgagatg

SEQ ID NO: 5 - DNA, 27 bp, Artificial
  synthetic PCR amplification Forward primer for beta3Gal-T2
tttggatccctgccaggcagagctgga

SEQ ID NO: 6 - DNA, 26 bp, Artificial
  synthetic PCR amplification Reverse primer for beta3Gal-T2
tttaagcttaatgtagtttacggtgg

SEQ ID NO: 7 - DNA, 28 bp, Artificial
  synthetic PCR amplification Forward primer for beta3GalNAc-T1
tttggatccagccttccccactacaatg

SEQ ID NO: 8 - DNA, 27 bp, Artificial
  synthetic PCR amplification Reverse primer for beta3GalNAc-T1
tttaagcttaataatggcatgtggtgt

SEQ ID NO: 9 - DNA, 27 bp, Artificial
  synthetic PCR amplification Forward primer for beta3Gal-T4
tttggatccttgggggaggagctgctg

SEQ ID NO: 10 - DNA, 27 bp, Artificial
  synthetic PCR amplification Reverse primer for beta3Gal-T4
tttgtcgactcagctctgaagccaggc

SEQ ID NO: 11 - DNA, 27 bp, Artificial
  synthetic PCR amplification Forward primer for beta3Gal-T6
tttggatccgaccccagggcgatgtcg

SEQ ID NO: 12 - DNA, 26 bp, Artificial
  synthetic PCR amplification Reverse primer for beta3Gal-T6
tttaagcttggctcaggggatgccct

SEQ ID NO: 13 - DNA, 22 bp, Artificial
  synthetic RT-PCR Forward primer for PCR of beta3Gal-T4
attcttgctgggagagccgaac

SEQ ID NO: 14 - DNA, 23 bp, Artificial
  synthetic RT-PCR Reverse primer for PCR of beta3Gal-T4
aagtacagaagaggcacttcctc

SEQ ID NO: 15 - DNA, 22 bp, Artificial
  synthetic RT-PCR Forward primer for PCR of beta3Gal-T5
ggaaacgaaagaggtggaccag

SEQ ID NO: 16 - DNA, 23 bp, Artificial
  synthetic RT-PCR Reverse primer for PCR of beta3Gal-T5
gagctcctccaatctgatgttca

SEQ ID NO: 17 - DNA, 25 bp, Artificial
  synthetic PCR amplification Forward primer for GlcNAc6ST2
tttgtcgacagccacaacatcagct

SEQ ID NO: 18 - DNA, 24 bp, Artificial
  synthetic PCR amplification Reverse primer for GlcNAc6ST2
tttaagcttagtggatttgctcag

SEQ ID NO: 19 - DNA, 26 bp, Artificial
  synthetic PCR amplification Forward primer for GlcNAc6ST2-pichia
tttcctaggtacagccacaacatcag

SEQ ID NO: 20 - DNA, 29 bp, Artificial
  synthetic PCR amplification Reverse primer for GlcNAc6ST2-pichia
tttgcggccgcttagtggatttgctcagg

SEQ ID NO: 21 - DNA, 24 bp, Artificial
  synthetic PCR amplification Forward primer for GlcNAc6ST1
tttgtcgactacaagtggcacaag

SEQ ID NO: 22 - DNA, 24 bp, Artificial
  synthetic PCR amplification Reverse primer for GlcNAc6ST1
tttgtcgaccccttttagagacgg

SEQ ID NO: 23 - DNA, 26 bp, Artificial
  synthetic PCR amplification Forward primer for GlcNAc6ST3
tttgtcgactcccggccagggccctc

SEQ ID NO: 24 - DNA, 26 bp, Artificial
  synthetic PCR amplification Reverse primer for GlcNAc6ST3
tttaagcttcagtcaggcgatgccca

SEQ ID NO: 25 - DNA, 23 bp, Artificial
  synthetic PCR amplification Forward primer for GlcNAc6ST4
tttgtcgacggcggccgcgacgg

SEQ ID NO: 26 - DNA, 26 bp, Artificial
  synthetic PCR amplification Reverse primer for GlcNAc6ST4
tttaagcttgggaggctacgtggcgc

SEQ ID NO: 27 - DNA, 26 bp, Artificial
  synthetic PCR amplification Forward primer for GlcNAc6ST5
tttgtcgactcccggccagggccctc

SEQ ID NO: 28 - DNA, 26 bp, Artificial
  synthetic PCR amplification Reverse primer for GlcNAc6ST5
tttaagcttgctacaactgtggcctc

SEQ ID NO: 29 - DNA, 26 bp, Artificial
  synthetic PCR amplification Forward primer for Gal6ST
tttgtcgacaccttcaccgccaagtc

SEQ ID NO: 30 - DNA, 26 bp, Artificial
  synthetic PCR amplification Reverse primer for Gal6ST
tttaagcttcacgagaaggggcggaa

SEQ ID NO: 31 - DNA, 26 bp, Artificial
  synthetic PCR amplification Forward primer for Ch6ST1
tttgtcgacatatcaagggtctcaga

SEQ ID NO: 32 - DNA, 27 bp, Artificial
  synthetic PCR amplification Reverse primer for Ch6ST1
tttaagcttctacgtgacccagaaggt

SEQ ID NO: 33 - protein, 310 aa, Homo sapiens
  beta1,3-galactosyltransferase-5 (beta3Gal-T5)
MAFPKMRLMYICLLVLGALCLYFSMYSLNPFKEQSFVYKKDGNFLKLPDTDCRQTPPFLVLLVTSSHKQLAERMAIRQTWGKERMVKGKQLKTFFLLGTTSSAAETKEVDQESQRHGDIIQKDFLDVYYNLTLKTMMGIEWVHRFCPQAAFVMKTDSDMFINVDYLTELLLKKNRTTRFFTGFLKLNEFPIRQPFSKWFVSKSEYPWDRYPPFCSGTGYVFSGDVASQVYNVSKSVPYIKLEDVFVGLCLERLNIRLEELHSQPTFFPGGLRFSVCLFRRIVACHFIKPRTLLDYWQALENSRGEDCPPV

SEQ ID NO: 34 - protein, 378 aa, Homo sapiens
  beta1,3-galactosyltransferase-4 (beta3Gal-T4)
MQLRLFRRLLLAALLLVIVWTLFGPSGLGEELLSLSLASLLPAPASPGPPLALPRLLIPNQEACSGPGAPPFLLILVCTAPENLNQRNAIRASWGGLREARGLRVQTLFLLGEPNAQHPVWGSQGSDLASESAAQGDILQAAFQDSYRNLTLKTLSGLNWAEKHCPMARYVLKTDDDVYVNVPELVSELVLRGGRWGQWERSTEPQREAEQEGGQVLHSEEVPLLYLGRVHWRVNPSRTPGGRHRVSEEQWPHTWGPFPPYASGTGYVLSASAVQLILKVASRAPLLPLEDVFVGVSARRGGLAPTQCVKLAGATHYPLDRCCYGKFLLTSHRLDPWKMQEAWKLVGGSDGERTAPFCSWFQGVLGILRCRAIAWLQS

SEQ ID NO: 35 - protein, 386 aa, Homo sapiens
  N-acetylglucose 6-O-sulfotransferase-2 (GlcNAc6ST-2)
MLLPKKMKLLLFLVSQMAILALFFHMYSHNISSLSMKAQPERMHVLVLSSWRSGSSFVGQLFGQHPDVFYLMEPAWHVWMTFKQSTAWMLHMAVRDLIRAVFLCDMSVFDAYMEPGPRRQSSLFQWENSRALCSAPACDIIPQDEIIPRAHCRLLCSQQPFEVVEKACRSYSHVVLKEVRFFNLQSLYPLLKDPSLNLHIVHLVRDPRAVFRSRERTKGDLMIDSRIVMGQHEQKLKKEDQPYYVMQVICQSQLEIYKTIQSLPKALQERYLLVRYEDLARAPVAQTSRMYEFVGLEFLPHLQTWVHNITRGKGMGDHAFHTNARDALNVSQAWRWSLPYEKVSRLQKACGDAMNLLGYRHVRSEQEQRNLLLDLLSTWTVPEQIH

(19) United States
(12) Patent Application Publication
(10) Pub. No.: US 2011/0306049 A1
(43) Pub. Date: Dec. 15, 2011

(54) METHOD FOR DETECTING GYNECOLOGIC CANCER

(75) Inventors: Katsuko Yamashita, Kanagawa (JP); Akira Seko, Kanagawa (JP); Daisuke Aoki, Tokyo (JP); Masaru Sakamoto, Tokyo (JP)

(73) Assignee: Tokyo Institute of Technology, Tokyo (JP)

(21) Appl. No.: 12/674,875

(22) PCT Filed: Aug. 22, 2008
(86) PCT No.: PCT/JP2008/065014

§ 371 Date: Feb. 23, 2010

(30) Foreign Application Priority Data
Aug. 24, 2007 (JP) 2007-219046
Mar. 13, 2008 (JP) 2008-063641

(51) Int. Cl. C12Q 1/68; C07K 16/40; G01N 33/574

(52) U.S. Cl. 435/6.12; 435/7.4; 530/389.7

(57) ABSTRACT

The object of the present invention is to provide a method for detecting gynecologic cancer and a kit for detecting the same.

The object can be solved by a method for detecting gynecologic cancer characterized in that β1,3-galactosyltransferase-5 and/or β1,3-galactosyltransferase-4, and GlcNAc 6-O-sulfotransferase-2 are analyzed. According to the detecting method, ovarian cancer and endometrial cancer patients can be detected at an early stage i.e. stages I and II, wherein patients have no subjective symptoms.

### TECHNICAL FIELD

The present invention relates to a method for detecting gynecologic cancers, particularly ovarian cancer or endometrial cancer, characterized in that β1,3-galactosyltransferase-5 and/or β1,3-galactosyltransferase-4, and GlcNAc 6-O-sulfotransferase are analyzed. The term “analyzing” or “analysis” as used herein includes a quantitative or semiquantitative measurement of an amount of a compound to be analyzed and a detection used to determine the presence or the absence of a compound to be analyzed.

### BACKGROUND ART

A gynecologic cancer includes, for example, endocervical cancer, endometrial cancer, ovarian cancer, valvar cancer, vaginal cancer, uterine sarcoma, and chorionic cancer (trophoblastic disease). In these many cancers, it is difficult to find the cancer by symptoms thereof at an early stage. However, five years survival rate is significantly increased by carrying out a surgical therapy such as operation at an early stage. Thus, it is desired to develop tumor marker capable of finding the gynecologic cancer at an early stage.

Ovarian cancer is developed in an ovary within the abdomen. Thus, even if the cancer has developed in patients, the patients with cancer hardly have subjective symptoms at an early stage. Therefore, in many cases, ovarian cancer patients are found in advanced stages such as stage III or stage IV after metastasizing. In such patients, even if a treatment is carried out, five years survival rate becomes low. For the treatment to the ovarian cancer patients, surgical therapy to operatively remove an ovary and a caul, irradiation therapy, and chemotherapy using anticancer drug are carried out.

At a less-advanced stage such as stage I or stage II in the ovarian cancer patients, it may be possible to cure ovarian cancer completely by operatively removing an ovary and surrounding tissues. On the contrary, at an advanced stage such as stage III or stage IV, metastasis of the ovarian cancer to the lymph node or the liver is developed. Since cancer tissue cannot be removed completely from the patients, irradiation therapy or chemotherapy is therefore carried out concurrently with the surgical therapy. However, it is difficult to completely prevent the metastatic cancer by irradiation therapy and chemotherapy, and thus, five years survival rate of such patients is extremely low in comparison with those of the patients which are found at stage I or II. Therefore, findings of the ovarian cancer patients at an early stage are desired.

As mentioned above, the ovarian cancer patients hardly have subjective symptoms at an early stage, and therefore many of the patients are found by means of diagnostic imaging technique using ultrasonic sound at an advanced stage.

As a diagnostic marker for detecting ovarian cancer, a measurement of glycoprotein, i.e. CA125, is most widely used. High positive rate of CA125 in serous adenocarcinoma is well known among four histological types of ovarian cancer, i.e. serous adenocarcinoma, mucinous adenocarcinoma, endometrioid adenocarcinoma, and clear cell adenocarcinoma. In addition, CA125 is a specific marker for ovarian cancer. Many of patients at advanced stage have positive CA125. However, positive rate of CA125 in the ovarian cancer patients at an early stage is low. Although an attempt of early diagnosis by means of CA125 assay in a health examination has been made in Europe and the United states, effectiveness thereof could not be revealed.

Endometrial cancer is developed in an endometrium within the uterus, and cannot be detected by cytodiagnosis which is carried out in a conventional gynecologic examination. The endometrial cancer patients are treated with surgical therapy by operatively removing cancer tissue, irradiation therapy, chemotherapy using anticancer drug, and hormonal therapy.

Stages of endometrial cancer may be classified into stage I to stage IV in accordance with the spread of cancer. Surgical therapy is applied in stages I to III, and then five years survival rate has been increased by carrying out the surgical therapy at an early stage. Therefore, findings of the endometrial cancer patients at an early stage are desired.

As a diagnostic marker of the endometrial cancer, CA125 or CA602 which is glycoprotein, or CA19-9 which is used as diagnostic marker for the pancreas cancer is used. However, the detection rates of the endometrial cancer patients by the diagnostic markers are extremely low, and thus, these diagnostic markers have no diagnostic benefit.

In various cancer cells and cancer tissues, increase or decrease of many proteins are reported. For example, it was reported that β1,3-galactosyltransferase-5 (hereinafter referred to as a β3Gal-T5), which is a glycosyltransferase involved in the synthesis of type1 (Galβ1-3GlcNAc) is expressed in colon cancer cells and pancreas cancer cells, and colon cancer tissues (patent reference 1 and non-patent reference 1). In addition, it has been reported that GlcNAc 6-O-sulfotransferase-2 (hereinafter referred to as a GlcNAc6ST-2), which is a sulfotransferase involved in the synthesis of L-selectin ligand, is expressed ectopically in mucinous colon cancer tissues (non-patent reference 2), and tissues of mucinous adenocarcinoma and clear cell adenocarcinoma (non-patent reference 3).

However, these reports merely disclose that the glycosyl/sulfotransferases are expressed in cancer cells or cancer tissues, but do not disclose usefulness of the glycosyl/sulfotransferases as a tumor marker in blood. Glycosyltransferases are not generally secreted to the outside of cells, and thus, it was thought that these glycosyltransferases are not useful as the tumor marker in bloods. Further, there is no report showing a correlation between β3Gal-T5 and ovarian cancer, β3Gal-T5 and endometrial cancer, or GlcNAc6ST-2 and endometrial cancer. Furthermore, as to β1,3-galactosyltransferase-4 (hereinafter referred to as a β3Gal-T4) which is one of β1,3-glycosyltransferase family, a correlation between β3Gal-T4 and ovarian cancer or endometrial cancer has not been reported.

### CITATION LIST

[patent reference 1] International Publication WO00/50608
[non-patent reference 1] Seko A., et. al. Cancer Research, 1996, (the United state), vol. 56, p. 3468-3473
[non-patent reference 2] Seko A., et al. Glycobiology, 2002, (the United state) vol. 12, p. 379-388
[non-patent reference 3] Kanoh A., et al. Glycoconjugate Journal, 2006, (Netherlands) vol. 23, p. 453-460

### DISCLOSURE OF THE INVENTION

### Problems to be Solved by the Invention

The present inventors have conducted intensive studies for a possibility of various intracellular proteins as a tumor marker, to find a detective marker capable of detecting gynecologic cancer patients, particularly ovarian cancer patients and endometrial cancer patients. As a result, the present inventors, surprisingly, found that β3Gal-T4 and/or β3Gal-T5, or GlcNAc6ST-2 were detected in the blood of the ovarian cancer patients at high rates. Particularly, the positive rate of β3Gal-T4 and/or β3Gal-T5 was high in all histological types of ovarian cancer, i.e. serous adenocarcinoma, mucinous adenocarcinoma, endometrioid adenocarcinoma, and clear cell adenocarcinoma. Furthermore, a scope of cancer cells secreting β3Gal-T4 and/or β3Gal-T5 does not fully overlap with that of cancer cells secreting GlcNAc6ST-2. Therefore, the present inventors found that a detective rate of ovarian cancer becomes significantly higher by a combination of an assay of β3Gal-T4 and/or β3Gal-T5, and an assay of GlcNAc6ST-2.

Further, the present inventors found that β3Gal-T4 and/or β3Gal-T5, or GlcNAc6ST-2 were detected in the blood of the endometrial cancer patients at high rates. In particular, the high positive rate of β3Gal-T4 and/or β3Gal-T5 in endometrial cancer patients is shown in comparison with CA125, CA602, or CA19-9. Furthermore, a scope of cancer cells secreting β3Gal-T4 and/or β3Gal-T5 does not fully overlap with one of cancer cells secreting GlcNAc6ST-2. Therefore, the present inventors found that a detective rate of endometrial cancer becomes significantly higher by the combination of the assay of β3Gal-T4 and/or β3Gal-T5, and the assay of GlcNAc6ST-2, and completed the present invention.

In this connection, the non-patent reference 3 discloses that GlcNAc6ST-2 is expressed ectopically in tissues of serous adenocarcinoma and clear cell adenocarcinoma. However, the non-patent reference 3 discloses in addition to the above information, that an expression of GlcNAc6ST-2 was not detected in endometrioid adenocarcinoma. That is, the non-patent reference 3 merely discloses that GlcNAc6ST-2 is expressed in tissues of serous adenocarcinoma and clear cell adenocarcinoma, and therefore the high detection rate of GlcNAc6ST-2 in blood of ovarian cancer patients could not be anticipated. In other words, it is unexpected for those skilled in the art that GlcNAc6ST-2 can be detected in blood of the patients with serous adenocarcinoma and clear cell adenocarcinoma at high rates, despite the fact that GlcNAc6ST-2 could not be detected in the tissues of serous adenocarcinoma and clear cell adenocarcinoma.

Accordingly, the object of the present invention is to provide a method for detecting gynecologic cancer, in particular, ovarian cancer or endometrial cancer, and a kit for detecting the same.

### Means for Solving the Problems

The present invention relates to a method for detecting gynecologic cancer characterized in that β1,3-galactosyltransferase-5 and/or β1,3-galactosyltransferase-4, and GlcNAc 6-O-sulfotransferase-2 are analyzed.

Further, the present invention relates to a method for detecting gynecologic cancer characterized in that β1,3-galactosyltransferase-5 and/or β1,3-galactosyltransferase-4 are analyzed.

Further, the present invention relates to a method for detecting gynecologic cancer characterized in that GlcNAc 6-O-sulfotransferase-2 is analyzed.

According to a preferable embodiment of the detecting method of the present invention, an antibody specifically binding to β1,3-galactosyltransferase-5 and β1,3-galactosyltransferase-4 is used.

According to a preferable embodiment of the detecting method of the present invention, an antibody specifically binding to GlcNAc 6-O-sulfotransferase-2 is used.

According to a preferable embodiment of the detecting method of the present invention, the gynecologic cancer is ovarian cancer or endometrial cancer.

As a sample to be tested in the method for detecting gynecologic cancer of the present invention, a biological sample isolated or derived from human body, for example, urine, blood, serum, plasma, spinal fluid, saliva, cell, tissue, organ, or preparation thereof (for example, biopsy specimen, particularly biopsy specimen of ovary) or the like may be used. The detecting method of the present invention can be used as a diagnostic method of gynecologic cancers, such as ovarian cancer or endometrial cancer, by detecting β1,3-galactosyltransferase-5 and/or β1,3-galactosyltransferase-4, and GlcNAc 6-O-sulfotransferase-2 from the samples to be tested isolated from human body.

Further, the present invention relates to a kit for detecting gynecologic cancer characterized by containing an antibody specifically binding to β1,3-galactosyltransferase-5 and β1,3-galactosyltransferase-4 or fragment thereof, and an antibody specifically binding to GlcNAc 6-O-sulfotransferase-2 or fragment thereof.

Further, the present invention relates to a kit for detecting gynecologic cancer characterized by containing an antibody specifically binding to β1,3-galactosyltransferase- and β1,3-galactosyltransferase-4 or fragment thereof.

Further, the present invention relates to a kit for detecting gynecologic cancer characterized by containing an antibody specifically binding to GlcNAc 6-O-sulfotransferase-2 or fragment thereof.

According to a preferable embodiment of the detecting kit of the present invention, the gynecologic cancer is ovarian cancer or endometrial cancer.

The present invention relates to a method for detecting ovarian cancer characterized in that β1,3-galactosyltransferase-5 and/or β1,3-galactosyltransferase-4, and GlcNAc 6-O-sulfotransferase-2 are analyzed.

Further, the present invention relates to a method for detecting ovarian cancer characterized in that β1,3-galactosyltransferase-5 and/or β1,3-galactosyltransferase-4 are analyzed.

Further, the present invention relates to a method for detecting ovarian cancer characterized in that GlcNAc 6-O-sulfotransferase is analyzed.

According to a preferable embodiment of the detecting method of the present invention, an antibody specifically binding to β1,3-galactosyltransferase-5 and β1,3-galactosyltransferase-4 is used.

According to a preferable embodiment of the detecting method of the present invention, an antibody specifically binding to GlcNAc 6-O-sulfotransferase-2 is used.

Further, the present invention relates to a kit for detecting ovarian cancer characterized by containing an antibody specifically binding to β1,3-galactosyltransferase-5 and β1,3-galactosyltransferase-4 or a fragment thereof, and an antibody specifically binding to GlcNAc 6-O-sulfotransferase-2 or a fragment thereof.

Further, the present invention relates to a kit for detecting ovarian cancer characterized by containing an antibody specifically binding to β1,3-galactosyltransferase-5 and β1,3-galactosyltransferase-4 or a fragment thereof.

Further, the present invention relates to a kit for detecting ovarian cancer characterized by containing an antibody specifically binding to GlcNAc 6-O-sulfotransferase-2 or a fragment thereof.

### Effects of the Invention

Hitherto, as a detective marker of ovarian cancer, CA125 or CA546 which are glycoproteins was widely used. According to the detecting method of the present invention, ovarian cancer patients can be detected at high rates in early stages i.e. stage I or II, in comparison with the above conventional detective marker of ovarian cancer. That is, according to the detecting method of the present invention, β3Gal-T4 and/or β3Gal-T5, or GlcNAc6ST-2, which are secreted from relatively-small cancer tissue, can also be detected, and therefore, ovarian cancer patients of stage I, who have no subjective symptoms, can be detected in a health examination. In addition, according to the detecting method wherein β3Gal-T4 and/or β3Gal-T5 is analyzed, of the present invention, mucinous adenocarcinoma, endometrioid adenocarcinoma, and clear cell adenocarcinoma can be detected in high rate in comparison with the conventional method, in particular, mucinous adenocarcinoma and clear cell adenocarcinoma, which are intractable and resistive to the chemotherapy, can be detected at high rates.

A detective rate of ovarian cancer can be significantly increased by a combination of a detecting method wherein β3Gal-T4 and/or β3Gal-T5 is analyzed of the present invention, and a detecting method wherein GlcNAc6ST-2 is analyzed of the present invention. In particular, in the patients at an early stage i.e. stage I or II of ovarian cancer, the combination thereof is significantly effective. In addition, in the patients of histological types of endometrioid adenocarcinoma and clear cell adenocarcinoma, the detection rate of ovarian cancer can be increased by the combination thereof.

As a detective marker of endometrial cancer, CA125, CA602, or CA19-9 was used. According to the detecting method of the present invention, endometrial cancer can be detected at high rates at an early stage i.e. stage I, in comparison with the above conventional detective markers. That is, according to the detecting method of the present invention, β3Gal-T4 and/or β3Gal-T5, or GlcNAc6ST-2, which are secreted from relatively-small amount of cancer tissue, can also be detected. Therefore, endometrial cancer, which may not be detected by cytodiagnosis in conventional gynecologic examination, can be detected.

Further, the detection rate of endometrial cancer can be significantly increased by the combination of a detecting method wherein β3Gal-T4 and/or β3Gal-T5 is analyzed of the present invention, and a detecting method wherein GlcNAc6ST-2 is analyzed of the present invention.

Furthermore, the detective method of the present invention can be also used in monitoring for a recurrence of cancer, and monitoring effects of irradiation therapy and chemotherapy.

### BRIEF DESCRIPTION OF DRAWINGS

FIG. 1 is photographs showing the specificity of monoclonal antibody and polyclonal antisera against 3Gal-T5. A: β3GalT-1 (lane 1), β3GalT-2 (lane 2), β3GalNAc-T1 (lane 3), β3Gal-T4 (lane 4), β3Gal-T5 (lane 5), and β3GalT-6 (lane 6) were subjected to electrophoresis, and stained by Sypro Orange (Invitrogen). B: Each protein was subjected to a western blotting method using a monoclonal antibody against β3Gal-T5. C: Each protein was subjected to a western blotting method using a polyclonal antisera against β3Gal-T5.

FIG. 2 is photographs showing the specificity of monoclonal antibody and polyclonal antisera against GlcNAc6ST-2. A: GlcNAc6ST1 (lane 1), GlcNAc6ST-2 (lane 2), GlcNAc6ST-3 (lane 3), GlcNAc6ST-4 (lane 4), GlcNAc6ST-5 (lane 5), Gal6ST (lane G), and Ch6ST1 (lane C) were subjected to electrophoresis, and stained by Sypro Orange (Invitrogen). B: Each protein was subjected to a Western blotting method using a monoclonal antibody against GlcNAc6ST-2. C: Each protein was subjected to Western blotting method using a polyclonal antisera against GlcNAc6ST-2.

FIG. 3A is a standard curve of β3Gal-T5 by a sandwich ELISA using a monoclonal antibody and polyclonal antisera against β3Gal-T5. FIG. 3B is a standard curve of GlcNAc6ST-2 by a sandwich ELISA using a monoclonal antibody and polyclonal antisera against GlcNAc6ST-2.

FIG. 4 is photographs wherein expression of mRNAs of β3Gal-T4 and β3Gal-T5 in five ovarian cancer cell lines i.e. ES-2 (lane 1), MCAS (lane 2), RMG-I (lane 3), RMG-II (lane 4), and RMUG-L (lane 5), were shown by RT-PCR.

FIG. 5A is a standard curve of β3Gal-T5 by a sandwich ELISA using a monoclonal antibody and polyclonal antisera against β3Gal-T5. As a diluent for standard substance, non-denatured β3Gal-T5 (E. coli), PBS with 0.1% Tween-20 (PBS-T) including 10% normal human serum was used. FIG. 5B is a standard curve of GlcNAc6ST-2 by a sandwich ELISA using a monoclonal antibody and polyclonal antisera against GlcNAc6ST-2. As a diluent for standard substance, non-denatured GlcNAc6ST-2 (E. coli), PBS with 0.1% Tween-20 (PBS-T) including 10% normal human serum was used.

### BEST MODE FOR CARRYING OUT THE INVENTION

### [1]A Method for Detecting Gynecologic Cancers

Gynecologic cancers include, for example, endocervical cancer, endometrial cancer, ovarian cancer, valvar cancer, vaginal cancer, uterine sarcoma, and chorionic cancer (trophoblastic disease). According to the detecting method of the present invention, these gynecologic cancer can be detected in patients. Particularly, ovarian cancer and endometrial cancer may be mentioned as cancers with a high detection rate. Therefore, as typical embodiments of the present invention, ovarian cancer and endometrial cancer are described hereinafter, but a gynecologic cancer in the present invention is by no means limited to the above two cancers.

### [1-1] Ovarian Cancer

The ovary is an oval organ about the size of thumb located at both sides of the uterus. Ovarian cancer develops therein, and is mostly an epithelial cancer. Ovarian cancer can be classified into stages I to IV, according to the cancer progression, as described bellow.

Stage I: The cancer is only inside the ovary. That is, the cancer is in one or both ovaries and is either on the outside of an ovary
Stage II: The cancer is in one or both ovaries and is extending into the pelvic tissues. That is, the cancer has spread (metastasized) to around an ovary (for example peritoneum such as the fallopian tubes, uterus, the rectum, or the bladder) within the pelvis.
Stage III: The cancer has spread (metastasized) (1) beyond the pelvis with peritoneal dissemination; (2) the retroperitoneum lymph nodes or inguinal lymph nodes; (3) the surface of liver; (4) the small intestine or colon histlogically. That is, the cancer has not been limited to around an ovary (peritoneum in the pelvic), but has spread (metastasized) to upper abdomen, or to retroperitoneum lymph nodes.
Stage IV: (1) the cancer has spread (metastasized) to the distant site; (2) the cancer cells are found in pleural fluid; (3) the cancer has spread (metastasized) to the inside of the liver. That is, the cancer has spread to the outside of the peritoneal cavity or the inside of the liver.

Due to an absence of subjective symptoms at an early stage, only about a third of ovarian cancer cases are found in stages I and II, and thus, two thirds thereof are found in stages III and IV. When ovarian cancer is found in stages III and IV, metastasis of ovarian cancer to an outside of cavitas pelvis has occurred.

Therefore, complete cure is difficult because the ovarian cancer found cannot be removed fully.

Although the histological types of ovarian cancer are varied, ovarian cancer may be classified mainly into serous adenocarcinoma, mucinous adenocarcinoma, endometrioid adenocarcinoma, and clear cell adenocarcinoma. Among the four histological types, it is considered that serous adenocarcinoma occurs most frequently. In most of the conventional ovarian cancer markers, such serous adenocarcinoma is detected. Further, in view of sensitivity to chemotherapy, it is considered that serous adenocarcinoma and clear cell adenocarcinoma exhibit a high resistance to anti tumor drug.

### [1-2] Endometrial Cancer

Endometrial cancer is developed in epithelium of uterus i.e. endometrium, and thus, is an epithelial cancer. Endometrial cancer can be classified into stages I to IV, according to the stage of cancer progression, as described bellow.

Stage I: The cancer is found in the uterine corpus only. That is, the cancer is not found in the uterine cervix or other parts.
Stage II: The cancer has spread from the uterus to the cervix. That is the cancer has not spread outside the uterus.
Stage III: the cancer has spread beyond the uterus and cervix, but has not spread beyond the pelvis. The cancer has spread (metastasized) to pelvic lymph nodes or periaortic lymph nodes.
Stage IV: The cancer has spread to other parts of the body beyond the pelvis; or bladder or bowel wall.

Endometrial cancer cannot be found by the cytodiagnosis for endocervical cancer in the conventional gynecologic examination. However, if the endometrial cancer cases can be found in stages 1 to III, surgical therapy by operation is applicable. Further, if the operation is carried out at an earlier stage, effect of therapy becomes higher.

[1-3] Analysis of β3Gal-T4 and/or β3Gal-T5

In the present invention, gynecologic cancer, in particular, ovarian cancer or endometrial cancer is detected by analyzing β3Gal-T4 and/or β3Gal-T5. Also, gynecologic cancer, in particular, ovarian cancer or endometrial cancer can be detected by analyzing β3Gal-T4. In addition, gynecologic cancer, in particular, ovarian cancer or endometrial cancer can be also detected by analyzing β3Gal-T5. Further, gynecologic cancer, in particular, ovarian cancer or endometrial cancer can be also detected by analyzing β3Gal-T4 and β3Gal-T5.

β3Gal-T4 belongs to β1,3-galactosyltransferase family, and synthesizes GD1a ganglioside, GM1 ganglioside, or GD1b ganglioside. While β3Gal-T5 exhibits a different substrate specificity from that of β3Gal-T4, and can synthesize Galβ1-3 GlcNAc structure.

As shown in Example 2, mRNAs of β3Gal-T4 and β3Gal-T5 are expressed in most cell lines derived from ovarian cancer. The scope of cell lines expressing mRNA of β3Gal-T4 does not fully overlap with that of β3Gal-T5. However, in all examined cell lines, mRNAs of β3Gal-T4 and/or β3Gal-T5 are expressed. In addition, the present inventors measured enzymatic activities of β3Gal-T4 and β3Gal-T5 in ovarian cancer tissues of five patients, and could detect the enzymatic activities of β3Gal-T4 and β3Gal-T5 in the tissues of all patients.

As shown in Example 1, specificities of the monoclonal antibody and the polyclonal antisera obtained by immunizing β3Gal-T5 were examined using other β1,3-galactosyltransferase. As a result, it was revealed that both β3Gal-T4 and β3Gal-T5 can be detected by an ELISA system by means of the use of the monoclonal antibody and the polyclonal antisera.

It is considered that ovarian cancer includes ovarian cancer cells secreting β3Gal-T4 and β3Gal-T5 to blood, ovarian cancer cells secreting β3Gal-T4 only to blood, and ovarian cancer cells secreting β3Gal-T5 only to blood. Therefore, it is considered that β1,3-galactosyltransferase, which is present in the sera of ovarian cancer patients and measured by the above ELISA system, may be β3Gal-T4 and/or β3Gal-T5.

Further, it is considered that endometrial cancer includes endometrial cancer cells secreting β3Gal-T4 and β3Gal-T5 to blood, endometrial cancer cells secreting β3Gal-T4 only to blood, and endometrial cancer cells secreting β3Gal-T5 only to blood. Therefore, it is considered that β1,3-galactosyltransferase, which is present in the sera of ovarian cancer patients and measured by the above ELISA system, may be β3Gal-T4 and/or β3Gal-T5.

In the present invention, the method for analyzing β3Gal-T4 and/or β3Gal-T5 is not particularly limited, so long as it can determine an amount of β3Gal-T4 and/or β3Gal-T5 quantitatively or semiquantitatively, or determine the presence or absence of β3Gal-T4 and/or β3Gal-T5. There may be mentioned, for example, an immunoassay using antibody against β3Gal-T4 and β3Gal-T5 or fragment thereof, such as enzyme immunoassay, latex agglutination assay, chemiluminescent immunoassay, fluorescence antibody technique, radioimmunoassay, immunoprecipitation technique, immunohistochemical assay, or Western blot analysis; a biochemical assay such as enzymatic analysis; and a molecular biological assay in which mRNA is measured.

When the immunoassay is used as the method for analyzing β3Gal-T4 and β3Gal-T5, the monoclonal or polyclonal antibody which binds to β3Gal-T4 and β3Gal-T5 may be used. Alternatively, a combination of a monoclonal or polyclonal antibody which specifically binds to β3Gal-T4, and a monoclonal or polyclonal antibody which specifically binds to β3Gal-T5, may be used.

When the immunoassay is used as the method for analyzing β3Gal-T4, the monoclonal or polyclonal antibody which binds to β3Gal-T4 may be used. In addition, the monoclonal or polyclonal antibody which binds to β3Gal-T4 and β3Gal-T5 may be used.

When the immunoassay is used as the method for analyzing β3Gal-T5, the monoclonal or polyclonal antibody which binds to β3Gal-T5 may be used. In addition, the monoclonal or polyclonal antibody which binds to β3Gal-T4 and β3Gal-T5 may be used.

The monoclonal antibody or polyclonal antibody may be produced according to known methods, except that β3Gal-T4 or β3Gal-T5 is used as an antigen for immunization. For example, the monoclonal antibody is prepared according to a cell fusion method of Kohler and Milstein (Kohler, G. and Milstein, C., Nature, 256, 495-497, 1975). In a preparation of polyclonal antibody, for example, β3Gal-T4 or β3Gal-T5 is conjugated to BSA or KLH. The conjugated β3Gal-T4 or β3Gal-T5, or unconjugated β3Gal-T4 or β3Gal-T5 is emulsified in an appropriate adjuvant (for example, Freund's complete adjuvant), and rabbits are routinely immunized intradermally by using them. If an antibody titer in blood is increased, blood is collected and can be used as an antiserum or an antibody purified by the known methods. In addition, a mouse or rabbit can be immunized with both β3Gal-T4 and β3Gal-5, to obtain a monoclonal antibody or polyclonal antibody which can bind to both β3Gal-T4 and β3Gal-T5.

When the biochemical assay is used as the method for analyzing β3Gal-T4 or β3Gal-T5, for example, the enzymatic activities of β3Gal-T4 or β3Gal-T5 can be measured in accordance with methods of Miyazaki et al. [Miyazaki et al., J. Biol. Chem., (1997) 272, 24794-24799] or Seko et al. [Seko et al., Cancer Res., (1996) 56, 3468-3473], whereby an amount of β3Gal-T4 or β3Gal-T5 or the presence or absence of β3Gal-T4 or β3Gal-T5 can be analyzed.

As an immunizing antigen for obtaining a monoclonal or polyclonal antibody against β3Gal-T5, a protein consisting of amino acid sequence of SEQ ID NO: 33, or a peptide consisting of a part thereof can be used. Specifically, an antigen purified from biological samples, a recombinant antigen prepared by a genetic engineering, or a partial peptide chemically synthesized can be used. In the case of the recombinant antigen expressed by using E. coli, yeast or the like, a fusion protein fused with other protein such as SOD or TrpE can be prepared, in order to express easily. In addition, a fusion protein fused with His-Tag can be prepared in order to purify easily. In particular, β3Gal-T5 wherein cytoplasmic domain and transmembrane domain are removed, is preferably expressed. For example, a polypeptide consisting of the 27th to 310th amino acids in the amino acid sequence of SEQ ID NO: 33, is preferably expressed as the recombinant protein. The monoclonal antibody and polyclonal antibody may be obtained by immunizing the recombinant polypeptide consisting of the 27th to 310th amino acids in the amino acid sequence of SEQ ID NO: 33 as an immunizing antigen.

An antibody binding to β3Gal-T4 and β3Gal-T5, which may be used in the method for detecting gynecologic cancers, particularly ovarian cancer or endometrial cancer of the present invention, is not limited, so long as it can at least bind β3Gal-T4 and β3Gal-T5. As the above antibody, there may be mentioned an antibody which also binds β1,3-galactosyltransferase other than β3Gal-T4 and β3Gal-T5, but preferably, an antibody which specifically binds β3Gal-T4 and β3Gal-T5.

An antibody binding to β3Gal-T4, which may be used in the method for detecting gynecologic cancer, particularly ovarian cancer or endometrial cancer of the present invention, is not limited, so long as it can at least bind β3Gal-T4. As the above antibody, there may be mentioned an antibody which also binds β1,3-galactosyltransferase other than β3Gal-T4, but preferably, an antibody which specifically binds β3Gal-T4.

An antibody binding to β3Gal-T5, which may be used in the method for detecting gynecologic cancers, particularly ovarian cancer or endometrial cancer of the present invention, is not limited, so long as it can at least bind β3Gal-T5. As the above antibody, there may be mentioned an antibody which also binds β1,3-galactosyltransferase other than β3Gal-T5, but preferably, an antibody which specifically binds β3Gal-T5.

As an immunizing antigen for obtaining a monoclonal or polyclonal antibody against β3Gal-T4, a protein consisting of amino acid sequence of SEQ ID NO: 34, or a peptide consisting of a part thereof can be used.

### [1-4] Analysis of GlcNAc6ST-2

In the present invention, gynecologic cancers, in particular, ovarian cancer or endometrial cancer are N-acetylglucosaminyl 6-O-sulfotransferase family, and transfers a sulfate group from an adenosine-3′-phosphate-5′phosphosulfate to a GlcNAc residue of various glycoprotein. In normal human, GlcNAc6ST-2 is expressed specifically in high endothelial cells, and is involved with a synthesis of L-selectin ligand.

The present inventors measured enzymatic activity of GlcNAc6ST-2 in tissues of five ovarian cancer patients, and could detect the enzymatic activity of GlcNAc6ST-2 in the tissues of all patients.

In the present invention, the method for analyzing GlcNAc6ST-2 is not particularly limited, so long as it can determine an amount of GlcNAc6ST-2 quantitatively or semiquantitatively, or determine the presence or absence of GlcNAc6ST-2. There may be mentioned, for example, an immunoassay using antibody against GlcNAc6ST-2 or fragment thereof, such as enzyme immunoassay, latex agglutination assay, chemiluminescent immunoassay, fluorescence antibody technique, radioimmunoassay, immunoprecipitation technique, immunohistochemical assay, or Western blot analysis; a biochemical assay such as enzymatic analysis; and a molecular biological assay in which mRNA is measured.

When the immunoassay is used as the method for analyzing GlcNAc6ST-2, the monoclonal or polyclonal antibody which binds to βGlcNAc6ST-2, may be used. The monoclonal antibody or polyclonal antibody may be produced according to known methods, except that GlcNAc6ST-2 is used as an antigen for immunization. For example, the monoclonal antibody is prepared according to the cell fusion method of Kohler and Milstein (Kohler, G. and Milstein, C., Nature, 256, 495-497, 1975). In a preparation of polyclonal antibody, for example, GlcNAc6ST-2 is conjugated to BSA or KLH. The conjugated GlcNAc6ST-2, or unconjugated GlcNAc6ST-2 is emulsified in an appropriate adjuvant (for example, Freund's complete adjuvant) and, rabbits are routinely immunized intradermally by using the same. If an antibody titer in blood is increased, blood is collected and can be used as an antisera or an antibody purified by known methods.

When the biochemical assay is used as the method for analyzing GlcNAc6ST-2, for example, the enzymatic activity of GlcNAc6ST-2 can be measured in accordance with method of Seko et al. [Seko et al., Glycobiology (2002) 12, 379-388], whereby an amount of GlcNAc6ST-2 or the presence or absence of GlcNAc6ST-2 can be analyzed.

As an immunizing antigen for obtaining a monoclonal or polyclonal antibody against GlcNAc6ST-2, a protein consisting of amino acid sequence of SEQ ID NO: 35, or a polypeptide consisting of a part thereof can be used. Specifically, an antigen purified from a biological sample, a recombinant antigen prepared by genetic engineering, or a partial peptide chemically synthesized can be used. In the case of the recombinant antigen expressed by using E. coli, yeast or the like, a fusion protein fused with other protein, such as SOD or TrpE can be prepared, in order to express easily. In addition, a fusion protein fused with His-Tag can be prepared, in order to purify easily. In particular, GlcNAc6ST-2, wherein cytoplasmic domain and transmembrane domain are removed, is preferably expressed. For example, a polypeptide consisting of the 28th to 386th amino acids in the amino acid sequence of SEQ ID NO: 35, is preferably expressed as a recombinant protein.

An antibody binding to GlcNAc6ST-2, which may be used in the method for detecting gynecologic cancers, particularly ovarian cancer or endometrial cancer of the present invention, is not limited, so long as it can at least bind GlcNAc6ST-2. As the above antibody, there may be mentioned an antibody which also binds GlcNAc6ST other than GlcNAc6ST-2, but preferably, an antibody which specifically binds GlcNAc6ST-2.

### [1-4] Immunological Analysis

When the enzyme immunoassay, for example a sandwich immunoassay, is used as the immunoassay, the sandwich immunoassay can be carried out according to the following procedure. The procedure of the sandwich immunoassay for GlcNAc6ST-2 is described in an illustration.

An antibody or an antibody fragment (a captured antibody or first antibody) binding to GlcNAc6ST-2 is immobilized to an appropriate insoluble carrier, such as a microtiter plate or a micro bead. Then the insoluble carrier is coated with an appropriate blocking agent, such as bovine serum albumin (BSA) or gelatin, to prevent a non-specific binding of a sample to the insoluble carrier. Thereafter, the sample which may contain GlcNAc6ST-2, and first reaction buffer are added to the microtiter plate or the micro bead. Then GlcNAc6ST-2 in the sample is brought into contact with the captured antibody, to perform a reaction (First reaction process). Then, unbinding antigen (GlcNAc6ST-2) or other substance in the sample is washed with an appropriate wash buffer, for example, phosphate buffer including detergent. Then, labeled antibody in which an antibody binding to the captured GlcNAc6ST-2 is conjugated to an enzyme such as horseradish peroxidase (HRP), is added to the whole, so as to bind the labeled antibody to the captured antigen (second reaction process), and whereby immune complex (i.e. the captured antibody-GlcNAc6ST-2-labeled antibody complex) is formed on the insoluble carrier such as the microtiter plate. An unbinding labeled antibody is washed with an appropriate wash buffer, and then a colorimetric substrate or a luminescent substrate for the enzyme of the labeled antibody is added. A detectable signal may be developed by a reaction in the enzyme and the substrate. Alternatively, a labeled antibody, which may bind to the second antibody, can be used instead of the labeled second antibody, in order to detect the signal.

As the enzyme for labeling the antibody, there may be mentioned a horseradish peroxidase (HRP), alkaline phosphatase, β-galactosidase, luciferase and the like. In addition, a luminescent material such as acridinium derivertives, a fluorescent material such as europium, or radioactive material such as I¹²⁵ can be used as a labeling material other than the enzyme. Further, a substance and a material for inducing luminescence are appropriately selected in accordance with the labeling material. The labeled antibody used in the present invention contains an antibody conjugated with a hapten, low molecular weight peptide, and a lectin which can be used for detecting the signal of antigen-antibody complex reaction.

In a sandwich immunoassay of β3Gal-T4 and/or β3Gal-T5, the sandwich immunoassay system may be constructed by replacing the antibody binding to GlcNAc6ST-2 with an antibody binding to β3Gal-T4 and/or β3Gal-T5 in the above sandwich immunoassay system of GlcNAc6ST-2.

If either the first antibody or the second antibody may specifically bind the protein to be analyzed, the other antibody, which may bind to a protein other than the protein to be analyzed or may exhibit cross-reactivity to the other protein, may be used in the sandwich ELISA. In the analysis of β3Gal-T4 and β3Gal-T5, for example, if either the first antibody or the second antibody may specifically bind β3Gal-T4 and β3Gal-T5, β3Gal-T4 and β3Gal-T5 can be analyzed specifically.

Further, in the analysis of GlcNAc6ST-2, if either the first antibody or the second antibody may specifically bind GlcNAc6ST-2, GlcNAc6ST-2 can be analyzed specifically. Furthermore, in the analysis of β3Gal-T4, if either the first antibody or the second antibody may specifically bind β3Gal-T4, β3Gal-T4 can be analyzed specifically. In addition, in the analysis of β3Gal-T5, if either the first antibody or the second antibody may specifically bind β3Gal-T5, β3Gal-T5 can be analyzed specifically.

The first antibody and the second antibody used in the sandwich immunoassay contains, but is not particularly limited to, a polyclonal antibody, monoclonal antibody, recombinant antibody, fragment thereof or the like. As the fragment of the antibody, there may be mentioned F(ab′)₂, Fab′, Fab, Fv, or the like. The fragment of the antibody may be obtained by conventional methods, for example, by digesting the antibody using a protease (such as pepsin, papain, or the like) and purifying the resulting fragments by standard polypeptide isolation and purification methods.

In the method for detecting gynecologic cancers, in particular, ovarian cancer or endometrial cancer of the present invention, as a sample to be tested, which is used for analyzing β3Gal-T4 and/or β3Gal-T5, or GlcNAc6ST-2, there may be mentioned urine, blood, serum, plasma, spinal fluid, saliva, cell, tissue, organ, or preparation thereof (for example, biopsy specimen, particularly biopsy specimen of ovary) or the like. The sample to be tested is preferably blood, serum, plasma, or biopsy specimen of ovary, more preferably blood, serum, or plasma (hereinafter referred to as a blood or the like). β3Gal-T4 and/or β3Gal-T5, or GlcNAc6ST-2 are not contained in the blood, serum, and plasma of normal humans. On the other hand, β3Gal-T4 and/or β3Gal-T5, or GlcNAc6ST-2 are released to the blood of ovarian cancer patients or endometrial cancer patients at an early stage of the cancer. Therefore, the blood or the like is appropriate as the sample to be tested for detecting ovarian cancer or endometrial cancer.

In the present invention, ovarian cancer or endometrial cancer can be detected by determining the presence or absence of β3Gal-T4 and/or β3Gal-T5, or GlcNAc6ST-2, or determining an amount of β3Gal-T4 and/or β3Gal-T5, or GlcNAc6ST-2 quantitatively or semiquantitatively. For example, when the biopsy specimen of the ovary or the uterus is used as the sample to be tested, the expression of β3Gal-T4 and/or β3Gal-T5, or GlcNAc6ST-2 can be determined by the immunohistochemical assay using the monoclonal or the polyclonal antibody, whereby it can be determined whether or not the sample is one of ovarian cancer, or the sample is one of endometrial cancer.

Further, when the blood or the like is used as the sample to be tested, blood is collected from a patient possibly suffering from ovarian cancer or endometrial cancer, then an amount of GlcNAc6ST-2 is measured using the collected blood or serum or plasma thereof. Then it can be determined whether or not the patient has ovarian cancer, or the patient has endometrial cancer by comparing the amount of GlcNAc6ST-2 of the patient with that of a normal human. More particularly, if the amount of GlcNAc6ST-2 in the patient is significantly high in comparison to that of a normal human, the patient can be diagnosed with ovarian cancer. In addition, as to β3Gal-T4 and β3Gal-T5, β3Gal-T4, or β3Gal-T5, it is possible to diagnose with ovarian cancer by comparing β3Gal-T4 and β3Gal-T5, β3Gal-T4, or β3Gal-T5 in the patients with that of a normal human.

For example, in the case of after-mentioned sandwich ELISA, the average of an amount of β3Gal-T4 and/or β3Gal-T5, or GlcNAc6ST-2 in a normal human is calculated, then the standard deviation (SD) thereof is calculated. In the present invention, the cut-off point for detecting the cancer patient is not limited, so long as each cancer can be detected by using the cut-off point. It is possible to define an average+SD, average+2SD, or average+3SD as cut-off point, and further, it is also possible that a sample having a value higher than average is diagnosed as positive one.

In the detection of gynecologic cancers, in particular, ovarian cancer or endometrial cancer of the present invention, most preferably the gynecologic cancer, in particular, ovarian cancer or endometrial cancer is specifically detected. However, if an early diagnosis of ovarian cancer by a blood test in a health examination is needed, preferably ovarian cancer patients or endometrial cancer patients can be detected preliminary. That is to say, it is preferable that the tumor marker can detect cancer patients as much as possible, among the ovarian cancer patients or endometrial cancer patients. According to the detecting method of the present invention, as shown in Examples below, the ovarian cancer patients (not including a patient supervening on other cancer) confirmed by the operation, or the confirmed endometrial cancer patients, can be detected at high rates. Therefore, the detecting method of the present invention is useful as a cancer marker.

### [2] Kit for Detecting Gynecologic Cancer

The kit for detecting gynecologic cancers, in particular, ovarian cancer or endometrial cancer of the present invention may contain the antibody specifically binding to β3Gal-T4 and β3Gal-T5, and the antibody specifically binding to GlcNAc6ST-2, or fragment thereof. As the antibody, a monoclonal antibody or a polyclonal antibody can be contained therein. The fragment of antibody is not particularly limited, so long as it can have specific binding ability to β3Gal-T4 and β3Gal-T5, or specific binding ability to GlcNAc6ST-2. For example, antibody fragment Fab, Fab′, F(ab′)₂, or Fv can be contained therein.

The detecting kit of the present invention may contain the antibody specifically binding to β3Gal-T4 and β3Gal-T5, or a fragment thereof; the antibody specifically binding to β3Gal-T4 or a fragment thereof; the antibody specifically binding to β3Gal-T5 or a fragment thereof; or the antibody specifically binding to GlcNAc6ST-2 or a fragment thereof.

Further, the detecting kit of the present invention may contain the antibody specifically binding to β3Gal-T4, or the antibody specifically binding to β3Gal-T5, instead of the antibody specifically binding to β3Gal-T4 and β3Gal-T5 or together with them.

The detecting kit of the present invention contains a desired form of antibody specifically binding to β3Gal-T4 and β3Gal-T5, or a fragment thereof, or desired form of antibody specifically binding to GlcNAc6ST-2, or a fragment thereof in accordance with the immunoassay used in the detecting kit.

For example, in a case of an immunoassay using the labeled antibody, such as enzyme immunoassay, chemiluminescent immunoassay, fluorescence antibody technique, or radioimmunoassay, the labeled antibody or antibody fragment conjugated by a labeling material can be contained therein. Specifically, the labeling material can include peroxidase, alkaline phosphatase, β-D-galactosidase, or glucose oxidase as an enzyme; fluorescein isothiocyanate, rare-earth metal chelate as a fluorescent material; ³H, ¹⁴C, or ¹²⁵I as a radioactive isotope; or biotin, avidin, or a chemiluminescent substance. In addition, in the case of an enzyme or a chemiluminescent substance, since they cannot develop a measurable signal by themselves, a substance corresponding to each enzyme or chemiluminescent substance is preferably contained therein.

### EXAMPLES

The present invention now will be further illustrated by, but is by no means limited to, the following Examples and Comparative Examples.

### Example 1

### Preparation of Anti β3Gal-T5 Antibody

### (A) Preparation of Soluble Form of β3Gal-T5 (Preparation of Antigen for Immunization)

### (A-1) Construction of β3Gal-T5 Expression Vector

A cDNA fragment encoding a peptide lacking cytoplasmic domain and transmembrane domain was amplified by PCR. The PCR was carried out by using a human colon cDNA library and the following Forward primer for b3Gal-T5 and Reverse primer for b3Gal-T5. The obtained cDNA fragment encodes a peptide consisting of the 27th to 310th amino acids in the amino acid sequence of β3Gal-T5.

Forward primer for b3Gal-T5:
(SEQ ID NO: 1)
5′-tttgtcgacAGTCTAAATCCTTTCAAAG-3′
Reverse primer for b3Gal-T5:
(SEQ ID NO: 2)
5′-tttaagcttCAGACAGGCGGACAATC-3′

In the above sequences, the sequences in lowercase letters contain appropriate restriction sites to be fused to an expression vector. The obtained cDNAs were inserted into a cloning site of pQE9 vector (QIAGEN GmbH, Hilden, Germany), then E. coli M15 cells were transformed with the resulting vector. The nucleotide sequences of the obtained plasmids were determined with a Prism 310 Genetic Analyzer (Applied Biosystems, Foster City, Calif.).

### (A-2) Expression and Purification of β3Gal-T5

Preparation of recombinant β3Gal-T5 was as follows. The overnight culture of transformed E. coli M15 cells were diluted by 1/100, added to 15 L of LB broth containing 100 μg/mL ampicillin and 25 μg/mL kanamycin, and grew at 30° C. for 3 hr with vigorous stirring. Isopropyl β-D-1-thiogalactopyranoside (hereafter, referred to as IPTG) having a final concentration of 0.1 mM was added to the culture and the expression of β3Gal-T5 was induced at 18° C. for 16 hr. The E. coli cells were collected with centrifugation and sonicated in Buffer A [200 mL of 6M guanidine-HCl, 20 mM potassium phosphate buffer (pH 8.0), 10 mM 2-mercaptoethanol, and 20 mM imidazole]. The obtained homogenate was allowed to stand at 25° C. for 16 hr. After centrifugation at 5,000 g for 20 min, the supernatant was applied to 3 mL of Ni-NTA agarose (QIAGEN GmbH) and allowed to stand for 30 min with occasional mixing. The resin was packed into a plastic column and washed with 60 mL of Buffer A. Thereafter, the recombinant protein was eluted with Buffer A containing 0.25M imidazole. The eluate was dialyzed against 8M urea-PBS, and concentrated with a Microcon YM-10 (Millipore Corp., Bedford, Mass.). Finally, 2.5 mg of the recombinant protein was obtained. The obtained protein was referred as denatured β3Gal-T5 (E. coli).

### (B) Preparation of Soluble Form of Non-Denatured β3Gal-T5

### (Preparation of Antigen for Screening)

Using the same transformed E. coli M15 as prepared in Example 1, non-denatured β3Gal-T5 was prepared. After induction with 0.1 mM IPTG in 300 mL E. coli cell culture, the cells were collected and sonicated in 25 mL of 20 mM potassium phosphate buffer (pH 8.0) (containing 0.3M NaCl and 10 mM imidazole). To the obtained homogenate, 2.5 mL of 5% Triton X-100 (v/v) was added and the mixture was allowed to stand on ice for 30 min. After centrifugation, 0.3 mL of Ni-NTA agarose was added to the supernatant, and the mixture was allowed to stand for 30 min with occasional mixing. The resin was packed into a plastic column and washed with 5 mL of Buffer B (20 mM potassium phosphate buffer (pH 8.0), 0.3M NaCl, and 20 mM imidazole) containing 0.5% Triton X-100, and further washed with another 5 mL of Buffer B. The recombinant protein was eluted with Buffer B containing 0.25M imidazole. The eluate was washed with PBS using a Microcon YM-10 (Millipore Corp., Bedford, Mass.) then concentrated. Among 0.36 mg of finally obtained proteins, recombinant β3Gal-T5 was approximately 10% yield, from estimation by SDS-PAGE analysis. It should be noted that this fraction contained substantial β3Gal-T5 activity. The obtained protein was referred to as denatured β3Gal-T5 (E. coli).

### (C) Preparation of Monoclonal and Polyclonal Antibodies

The monoclonal and the polyclonal antibodies against β3Gal-T5 were commercially prepared by Medical and Biological Laboratories Co., Ltd. (Nagoya, Japan). The monoclonal antibodies were prepared as follows. The above denatured β3Gal-T5 (E. coli) was injected three times into the footpads of four mice at three day intervals. In accordance with the conventional method, mouse spleen cells were fused with mouse myeloma cells, then the screening of hybridomas for producing antibodies were carried out by using the non-denatured β3Gal-T5 (E. coli) as the antigen. A hybridoma for producing monoclonal antibody which binds to the non-denatured β3Gal-T5 was cloned. In accordance with the conventional method, the hybridoma was intraperitoneally-inoculated to five mice, to obtain 25 mL of ascites. The ascitic fluid was purified with Sephacryl S-300 gel filtration (1.4×68 cm; equilibrated and eluted with TBS: GE Healthcare) to obtain 21 mg of β3Gal-T5 monoclonal antibody.

In the case of polyclonal antibody, the non-denatured β3Gal-T5 (E. coli) was injected six times into a rabbit at one week intervals to obtain β3Gal-T5 polyclonal antisera.

### (D) Identification of Specificities in Monoclonal and Polyclonal Antibodies

To identify the specificities in the resulting monoclonal and the polyclonal antibodies with respect to β3Gal-T5, reactivity with respect to each of β3GalT-1, β3GalT-2, β3Gal-T4, β3GalT-6, and β3GalNAc-T1 was examined, respectively. First, the recombinant protein for each of β3GalT-1, β3GalT-2, β3Gal-T4, β3GalT-6, and β3GalNAc-T1 was prepared. The step (A-1) was repeated to construct an expression vector, except that the following primers were used as a primer for β3GalT-1. On the other hand, for β3GalT-2, β3Gal-T4, β3GalT-6, and β3GalNAc-T1, the step (A-1) was repeated to construct an expression vector, except that a Super Script human brain cDNA library was used as cDNA library instead of a human colon cDNA library, and the following primers were used as primer.

Forward primer for β3Gal-T1:
(SEQ ID NO: 3)
5′-tttggatccAGTATAACTCGCCCTACTT-3′
Reverse primer for β3Gal-T1:
(SEQ ID NO: 4)
5′-tttaagcttCCTAACATCTGAGATG-3′
Forward primer for β3Gal-T2:
(SEQ ID NO: 5)
5′-tttggatccCTGCCAGGCAGAGCTGGA-3′
Reverse primer for β3Gal-T2:
(SEQ ID NO: 6)
5′-tttaagcttAATGTAGTTTACGGTGG-3′
Forward primer for β3GalNAc-T1:
(SEQ ID NO: 7)
5′-tttggatccAGCCTTCCCCACTACAATG-3′
Reverse primer for β3GalNAc-T1:
(SEQ ID NO: 8)
5′-tttaagcttAATAATGGCATGTGGTGT-3′
Forward primer for β3Gal-T4:
(SEQ ID NO: 9)
5′-tttggatccTTGGGGGAGGAGCTGCTG-3′
Reverse primer for β3Gal-T4:
(SEQ ID NO: 10)
5′-tttgtcgacTCAGCTCTGAAGCCAGGC-3′
Forward primer for β3Gal-T6:
(SEQ ID NO: 11)
5′-tttggatccGACCCCAGGGCGATGTCG-3′
Reverse primer for β3Gal-T6:
(SEQ ID NO: 12)
5′-tttaagcttGGCTCAGGGGATGCCCT-3′

Each recombinant protein was prepared by carrying out step (A-2) with few modifications. Concretely, one hundredth part of each overnight culture of the transformed M15 cells was added to 15 mL of LB broth containing the antibiotics, and cultured at 30° C. for 3 hr. IPTG having a final concentration of 0.1 mM was added to the cultures and the expression was induced at 18° C. for 16 hr. The E. coli cells were collected and sonicated in 10 mL of Buffer A. The homogenates were allowed to stand at 25° C. for 16 hr. After centrifugation, the supernatants were added to 0.5 mL of Ni-NTA agarose (QIAGEN GmbH) and were allowed to stand for 30 min. The resins were washed with 10 mL of Buffer A, and the recombinant proteins were eluted with Buffer A containing 0.25M imidazole. As a result, the yields for β3GalT-1, β3GalT-2, β3Gal-T4, β3GalT-6, and (3GalNAc-T1 were 0.03 mg, 0.03 mg, 0.06 mg, 0.70 mg, and 1.8 mg, respectively.

Then the reactivities were identified by using Western blotting analysis. 300 ng of each obtained protein was loaded with polyacrylamide gel, and transferred to a nitrocellulose membrane. After blocking, the membrane was incubated with anti β3Gal-T5 monoclonal antibody (1 μg/mL). The membrane was washed, and further incubated with HRP labeled anti mouse IgG/IgM rabbit antibody (0.3 μg/mL: Jackson ImmunoReseach). The chemiluminescence detection was carried out using ECL Western blot detection reagent (GE Healthcare).

For the reactivities on in polyclonal antisera, 50 ng of each protein was loaded, and a nitrocellulose membrane was incubated with the polyclonal antisera diluted by a factor of 1000. After washing, the membrane was incubated with a HRP labeled anti rabbit Ig antibody. Then Chemiluminescence detection was carried out as is in the case of the monoclonal antibody.

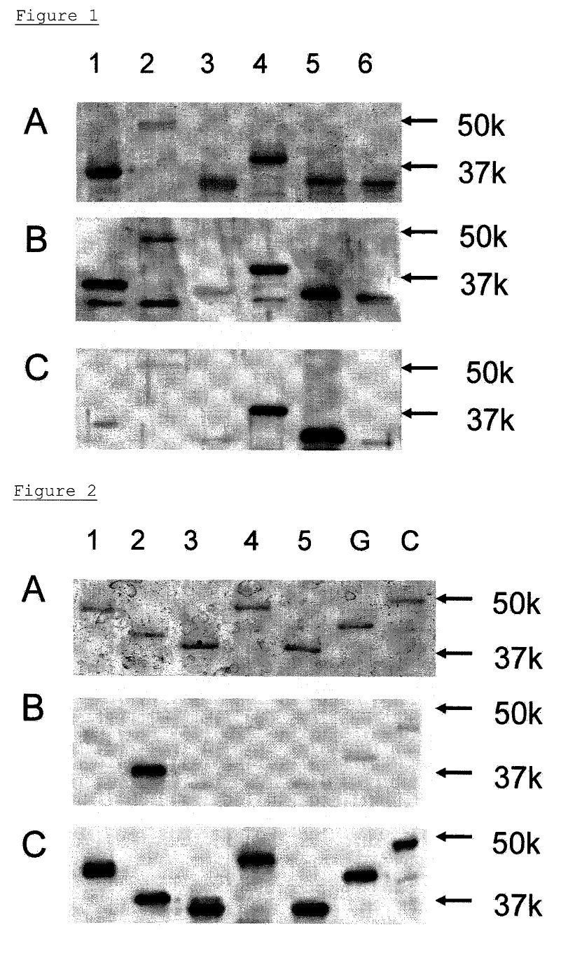

As shown in FIG. 1, the monoclonal antibody showed an intense reactivity to β3Gal-T5. Further, it also showed reactivities for β3GalT-1 and β3Gal-T4. On the other hand, the polyclonal antibody showed an intense reactivity for β3Gal-T5, as well as reactivity for β3Gal-T4.

### Example 2

### Expression of mRNAs of β3Gal-T4 and β3Gal-T5 in Ovarian Cancer Cells

The Expression of mRNAs of β3Gal-T4 and β3Gal-T5 in ovarian cancer cells was investigated by RT-PCR using five different types of ovarian cancer cells. ES-2 cells (ovarian clear cell carcinoma) were purchased from ATCC, and MCAS cells (ovarian mucinous cystadenocarcinoma), RMG-I cells (ovarian mesonephroid adenocarcinoma), RMG-II cells (ovarian mesonephroid adenocarcinoma), and RMUG-L cells (ovarian mucinous cystadenocarcinoma) were purchased from Human Science Research Resources Bank (Osaka, Japan).

The harvested cells were lysed using ISOGEN (Nippon Gene, Japan) to isolate total RNAs. The cDNAs were synthesized from the obtained RNAs by using oligo(dT) primers and SuperScript 111 (Invitrogen). The amounts of cDNAs were normalized by those of β-actin (Seko et al., 2002).

The following primers were added to the cDNAs, and the incubation was carried out at 95° C. for 2 min. Afterward, the expression of β3Gal-T4 and β3Gal-T5 was examined by PCR. PCR was performed using GoTaq DNA polymerase (Promega) in a volume of 20 μL for 29 cycles of 95° C. for 30 seconds, 62° C. for 1 minute, and 72° C. for 2 minutes.

Forward primer for PCR of β3Gal-T4:

| | |
|---|---|
| 5′-ATTCTTGCTGGGAGAGCCGAAC-3′ | (SEQ ID NO: 13) |

Reverse primer for PCR of β3Gal-T4:

| | |
|---|---|
| 5′-AAGTACAGAAGAGGCACTTCCTC-3′ | (SEQ ID NO: 14) |

Forward primer for PCR of β3Gal-T5:

| | |
|---|---|
| 5′-GGAAACGAAAGAGGTGGACCAG-3′ | (SEQ ID NO: 15) |

Reverse primer for PCR of β3Gal-T5:

| | |
|---|---|
| 5′-GAGCTCCTCCAATCTGATGTTCA-3′ | (SEQ ID NO: 16) |

As shown in FIG. 4, mRNA of β3Gal-T4 was expressed in each of ES-2 cells, MCAS cells, RMG-I cells, and RMUG-L cells. On the other hand, mRNA of β3Gal-T5 was expressed in each of ES-2 cells, RMG-I cells, RMG-II cells, and RMUG-L cells.

mRNA of β3Gal-T4 and mRNA of β3Gal-T5 were expressed in four ovarian cancer cell strains among the five ovarian cancer cell strains. Further, in the five ovarian cancer cell strains, either one of mRNA of β3Gal-T4 or mRNA of β3Gal-T5 was expressed.

### Example 3

### Preparation of GlcNAc6ST-2 Antibody

(A) Preparation of Soluble form of GlcNAc6ST-2 (Preparation of Antigen for Immunization)

### (A-1) Construction of GlcNAc6ST-2 Expression Vector

A cDNA fragment encoding a peptide lacking cytoplasma and transmembrane domains was amplified by PCR. The PCR was carried out using a full-length cDNA prepared previously (Glycobiology, 2002, 12, p. 379-388) and the following Forward primer for GlcNAc6ST2 and Reverse primer for GlcNAc6ST2. The obtained cDNA fragment encodes a peptide consisting of the 28th to 386th amino acids in the amino acid sequence of GlcNAc6ST-2.

| | |
|---|---|
| Forward primer for GlcNAc6ST2: | |
| 5′-tttgtcgacAGCCACAACATCAGCT-3′ | (SEQ ID NO: 17) |
| Reverse primer for GlcNAc6ST2: | |
| 5′-tttaagcttAGTGGATTTGCTCAG-3′ | (SEQ ID NO: 18) |

In the above sequences, the sequences in lowercase letters contain appropriate restriction sites to be fused to an expression vector. The obtained cDNAs were inserted into a cloning site of pQE9 vector (QIAGEN GmbH, Hilden, Germany), and then, E. coli M15 cells were transformed with the resulting vector. The nucleotide sequences of the obtained plasmids were determined with a Prism 310 Genetic Analyzer (Applied Biosystems, Foster City, Calif.).

### (A-2) Expression and Purification of GlcNAc6ST-2

The step (A-2) in Example 1 was repeated to obtain 3.5 mg of GlcNAc6ST-2, except that the culture for expression induction by IPTG was carried out at 1.25 L using a GlcNAc6ST-2 expression vector instead of a β3Gal-T5 expression vector. The obtained proteins were referred as E. coli GlcNAc6ST-2.

(B) Preparation of Soluble form of GlcNAc6ST-2 in Pichia pastoris (Preparation of Antigen for Screening)

GlcNAc6ST-2 was prepared by using yeasts for screening monoclonal antibodies. A cDNA fragment encoding a peptide laking cytoplasma and transmembrane domains was amplified by PCR. The PCR was carried out by using a full-length cDNA prepared previously (Glycobiology. 12, p. 379-388, 2002) and the following Forward primers for GlcNAc6ST2-pichia and Reverse primer for GlcNAc6ST2-pichia.

Forward primer for GlcNAc6ST2-pichia:
(SEQ ID NO: 19)
5′-tttcctaggTACAGCCACAACATCAG-3′
Reverse primer for GlcNAc6ST2-pichia:
(SEQ ID NO: 20)
5′-tttgcggccgcTTAGTGGATTTGCTCAGG-3′

The obtained cDNA was inserted into cloning sites appropriate for pPIC9-His vector and the nucleotide sequence was determined with a Prism 310 Genetic Analyzer. Production and purification of the recombinant GlcNAc6ST-2 was carried out in accordance with the known method (Glycobiology 15 p. 943-951, 2005). From 500 mL of a culture in buffered methanol-complex medium, 300 μg of the recombinant protein was obtained. It should be noted that this fraction contained substantial GlcNAc6ST-2 activity. The obtained protein was referred to as yeast GlcNAc6ST-2.

### (C) Preparation of Monoclonal and Polyclonal Antibodies

Step (C) in Example 1 was repeated to obtain a strain of hybridoma for producing monoclonal antibody and to obtain polyclonal antisera, except that E. coli GlcNAc6ST-2 cells were used as immunogen and that yeast GlcNAc6ST-2 was used as screening antigen for the monoclonal antibody. The hybridoma was intraperitoneally-inoculated into five mice, to obtain 13 mL of ascites. The obtained ascetic fluid was purified with protein A-Sepharose affinity chromatography (0.9×7.9 cm; equilibrated with PBS: GE Healthcare) to obtain 12.6 mg of antibody.

### (D) Identification of Specificities in Monoclonal and Polyclonal Antibodies

To identify the specificities in the resulting monoclonal and polyclonal antibodies with respect to GlcNAc6ST-2, the reactivity with respect to each of GlcNAcc6ST1, GlcNAcc6ST3, GlcNAcc6ST4, GlcNAcc6ST5, Gal6ST, and Ch6ST1 was examined, respectively. First, the recombinant protein for each of GlcNAcc6ST1, GlcNAcc6ST3, GlcNAcc6ST4, GlcNAcc6ST5, Gal6ST, and Ch6ST1 was prepared. Then, step (A-1) in Example 1 was repeated to construct an expression vector, except that the following primers were used.

Forward primer for GlcNAc6ST1:
(SEQ ID NO: 21)
5′-tttgtcgacTACAAGTGGCACAAG-3′
Reverse primer for GlcNAc6ST1:
(SEQ ID NO: 22)
5′-tttgtcgacCCCTTTTAGAGACGG-3′
Forward primer for GlcNAc6ST3:
(SEQ ID NO: 23)
5′-tttgtcgacTCCCGGCCAGGGCCCTC-3′
Reverse primer for GlcNAc6ST3:
(SEQ ID NO: 24)
5′-tttaagcttCAGTCAGGCGATGCCCA-3′
Forward primer for GlcNAc6ST4:
(SEQ ID NO: 25)
5′-tttgtcgacGGCGGCCGCGACGG-3′
Reverse primer for GlcNAc6ST4:
(SEQ ID NO: 26)
5′-tttaagcttGGGAGGCTACGTGGCGC-3′
Forward primer for GlcNAc6ST5:
(SEQ ID NO: 27)
5′-tttgtcgacTCCCGGCCAGGGCCCTC-3′
Reverse primer for GlcNAc6ST5:
(SEQ ID NO: 28)
5′-tttaagcttGCTACAACTGTGGCCTC-3′
Forward primer for Gal6ST:
(SEQ ID NO: 29)
5′-tttgtcgacACCTTCACCGCCAAGTC-3′
Reverse primer for Gal6ST:
(SEQ ID NO: 30)
5′-tttaagcttCACGAGAAGGGGCGGAA-3′
Forward primer for Ch6ST1:
(SEQ ID NO: 31)
5′-tttgtcgacATATCAAGGGTCTCAGA-3′
Reverse primer for Ch6ST1:
(SEQ ID NO: 32)
5′-tttaagcttCTACGTGACCCAGAAGGT-3′

Step (D) in Example 1 for the expression and purification of β3GalT-1 and so on, was repeated to obtain each recombinant protein, except that 15 mL of culture was used for the expression induction by IPTG. The yields for GlcNAcc6ST1, GlcNAcc6ST3, GlcNAcc6ST4, GlcNAcc6ST5, Gal6ST, and Ch6ST1 were 0.20 mg, 1.2 mg, 0.26 mg, 0.83 mg, 0.26 mg, and 0.13 mg, respectively.

TABLE 1

| | | Number of | | | GN6ST2 + | | |
|---|---|---|---|---|---|---|---|
| | | samples | GN6ST2 | β3GalT5 | β3GalT5 | CA602/125 | CA564 |
| Disease | Disease states I to II | 31 | 74.2 | 87.1 | 96.8 | 73.1 | 70.8 |
| stages | Disease states III to IV | 29 | 58.6 | 93.1 | 93.1 | 96.3 | 95.5 |
| Histol- | serious | 18 | 44.4 | 88.9 | 88.9 | 93.8 | 92.9 |
| ogical | adenocarcinoma | | | | | | |
| types | mucinous | 10 | 70.0 | 100 | 100 | 71.4 | 85.7 |
| | adenocarcinoma | | | | | | |
| | endometrioid | 13 | 76.9 | 84.6 | 100 | 77.8 | 83.3 |
| | adenocarcinoma | | | | | | |
| | clear cell | 16 | 75.0 | 87.5 | 93.8 | 86.7 | 69.2 |
| | adenocarcinoma | | | | | | |
| | Serous + Endometrioid | 31 | 58.1 | 87.1 | 93.5 | 88.0 | 90.0 |
| | Mucinous + Clear cell | 26 | 73.1 | 92.3 | 96.2 | 81.8 | 75.0 |

* Positive rates are given in %.

Then the western blotting method was performed. The western blotting step (D) in Example 1 was repeated, except that 200 ng of each of the antigens for the electrophoresis was used.

As shown in FIG. 2, the monoclonal antibody showed an intensive reactivity only to GlcNAc6ST-2. On the other hand, the polyclonal antisera showed reactivity to all antigens.

### Example 4

### Construction of Sandwich ELISA of β3Gal-T5 and GlcNAc6ST-2

Construction of an immunoassay system for β3Gal-T5 and GlcNAc6ST-2 were performed by a sandwich ELISA method using the monoclonal antibody and polyclonal antisera obtained by immunizing the β3Gal-T5, and by using the monoclonal antibody and polyclonal antisera obtained by immunizing the GlcNAc6ST-2.

### (A) β3Gal-T5 Detection System

50 μL of b3Gal-T5 monoclonal antibody, which had been diluted to a concentration of 1 μg/mL with a PBS, was added to 96-well black high-binding plates (Corning Inc., Corning, N.Y.), and was immobilized at 4° C. for 16 hr. The surface of each well was blocked with PBS containing 1% BSA at 25° C. for 1 hr. The non-denatured β3Gal-T5 (E. coli) as reference materials, was diluted to a concentration of 250 ng/mL, 125 ng/mL, 62.5 ng/mL, 16 ng/mL, and 4.0 ng/mL with a diluent wherein 1% BSA had been added to PBS-0.1% Tween-20 (PBS-T). Then 50 μL of the non-denatured β3Gal-T5 (E. coli) was added to the wells, and incubated at 25° C. for 2 hr. After washing with PBS-T three times, 50 μL of β3Gal-T5 polyclonal antisera diluted by a factor of 1000 was added and incubated at 25° C. for 1.5 hr. After washing with PBS-T three times, donkey HRP-labeled anti-rabbit Ig antibody (0.3 μg/mL: GE Healthcare) diluted with PBS-T was added and incubated at 25° C. for 1 hr. The wells were washed with PBS-T four times, and 100 μL of SuperSignal ELISA Pico Chemiluminescent Substrate (PIERCE, Rockford, Ill.) was added and chemiluminescent quantification was carried out by using a Plate CHAMELEON V (HIDEX Oy, Turku, Finland).

### (B) GlcNAc6ST-2 Detection System

Step (A) was repeated to construct a sandwich ELISA of GlcNAc6ST-2, except that the monoclonal antibody having a concentration of 3 μL/mL with respect to GlcNAc6ST-2 was immobilized, the polyclonal antisera with respect to GlcNAc6ST-2 was diluted by a factor of 3000, and GlcNAc6ST-2 (E. coli) was used as reference material.

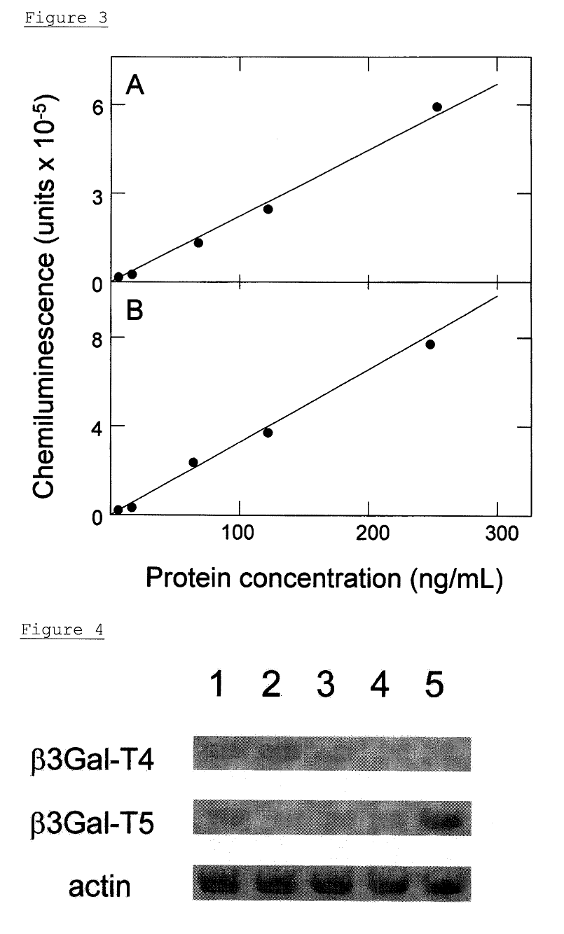

As shown in FIG. 3, a good linear standard curve for each of the β3Gal-T5 detection system and the GlcNAc6ST-2 detection system were obtained.

### (C) Improvement in a Standard Curve for the β3Gal-T5 Detection System

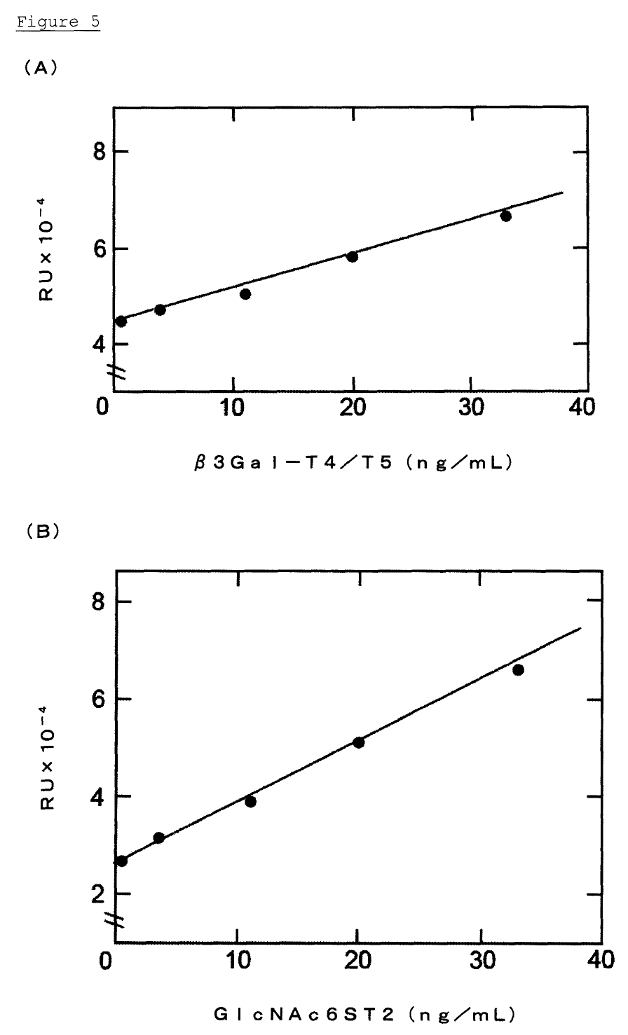

Step (A) was repeated to obtain the standard curve for β3Gal-T5, except that non-denatured β3Gal-T5 (E. coli) was diluted using PBS-0.1% Tween-20 (PBS-T) wherein normal human serum 10% was added, instead of using PBS-0.1% Tween-20 (PBS-T). The results are shown in FIG. 5A. At low concentrations of β3Gal-T5, the linearity of the standard curve was improved. Other than the normal human serum, the use of sera of bovine, horse, sheep, goat or the like showed the same effects as obtained in the normal human serum.

### (D) Improvement in a Standard Curve for GlcNAc6St-2 Detection System

Step (B) was repeated to obtain the standard curve for GlcNAc6ST-2, except that non-denatured GlcNAc6ST-2 (E. coli) was diluted using PBS-0.1% Tween-20 (PBS-T) wherein normal human serum 10% was added, instead of using PBS-0.1% Tween-20 (PBS-T). The results are shown in FIG. 5A. At low concentrations of GlcNAc6ST-2, the linearity of the standard curve was improved.

### Example 5

### Measurement in Sera of Ovarian Cancer Patients Using β3Gal-T5 Detection System

Sera of 60 cases of ovarian cancer patients and sera of 8 samples of healthy adult women were measured using the β3Gal-T5 detection system. The sera of the ovarian cancer patients to be used in the measurement were obtained from ovarian cancer patients of stages I to IV which were confirmed by abdominal operation. In the 57 cases, it was possible to determine the histological types of ovarian cancer by using histological examination. Any cancer developments other than ovarian cancer were not confirmed in these ovarian cancer patients.

Step (A) in Example 4 was repeated, except that the sera of patients and of healthy persons diluted with PBS-T by a factor of 10 were used instead of non-denatured β3Gal-T5 (E. coli). The amount of β3Gal-T5 in the sera was determined by the standard curve (A) in FIG. 3. The value of an average+2SD in healthy adult women was defined as a cut-off point, and it was determined whether the sera of each patient was positive or negative.

In the examples, the monoclonal antibody and the polyclonal antisera were obtained by immunizing β3Gal-T5 in (D) of Example 1. The reactivities of the monoclonal antibody and the polyclonal antisera with respect to other β1,3-galactosyltransferases were examined. As a result, it is possible to detect both β3Gal-T4 enzyme and β3Gal-T5 enzyme by ELISA using the monoclonal antibody and the polyclonal antisera.

### Example 6

### Measurement in Sera of Ovarian Cancer Patients by Using GlcNAc6St-2 Detection System

Sera of 60 cases of ovarian cancer patients and sera of 8 samples of healthy adult women as tested in Example 5, were measured by using GlcNAc6ST-2 detection system.

Step (B) of Example 4 was repeated, except that the sera of the patients diluted with PBS-T by a factor of 10 were used, instead of GlcNAc6ST-2 (E. coli). The amount of GlcNAc6ST-2 in the sera was determined by the standard curve (B) in FIG. 3. The value of an average+2SD in healthy adult women was defined as a cut-off point, and it was determined whether the serum of each patient was positive or negative.

### Comparative Example 1

In the sera of 60 cases of the ovarian cancer patients and in the sera of 8 samples of the healthy adult women as tested in Example 5, CA125 which is known as a conventional marker for ovarian cancer was measured.

The measurement of CA125 was carried out by using a kit of ECLusys reagent CA125II (Roche Diagnostics), in accordance with the attached protocol.

### Comparative Example 2

In the sera of 60 cases of the ovarian cancer patients and in the sera of 8 samples of the healthy adult women as tested in Example 5, CA546 which is known as a conventional marker for ovarian cancer was measured.

The measurement of CA546 was carried out by using a kit of PLATE Kainos CA546 reagent (Kainos Laboratories, Inc.) in accordance with the attached protocol.

### Analytic Example 1

The 60 tested cases of ovarian cancer were classified into disease stages or histological types of ovarian cancer and positive rates obtained from Examples 4 and 5, and Comparative Example 1 and 2 were analyzed. The results are shown in Table 1.

In the early ovarian cancer of stages I and II, the positive rate detected using β3Gal-T5 detection system was 87.1%, and the positive rate detected by using GlcNAc6ST-2 detection system was 74.2%. It was possible to detect ovarian cancer at high rate in comparison with the positive rates detected using CA125 and CA546. Further, in the measurement by using β3Gal-T5 detection system and/or GlcNAc6ST-2 detection system, 96.8% of samples ware positive. Thus, it was possible to detect the early ovarian cancer patients at high rate, by combining the immunological methods of β3Gal-T5 and GlcNAc6ST-2.

Moreover, if ovarian cancers are classified into each of the histological types, the positive rate for mucinous adenocarcinoma was 100%, the positive rate for endometrioid adenocarcinoma was 84.6%, and the positive rate for clear cell adenocarcinoma was 87.5%, in accordance with the immunological method by using β3Gal-T5 detection system. These positive rates were higher than those obtained by CA125 or CA546. In particular, the immunological method using β3Gal-T5 detection system was able to detect the mucinous adenocarcinoma and clear cell adenocarcinoma at high rate, even thought these cancers are usually intractable and resistive to the chemotherapies.

In addition thereto, the positive rate for endometrioid ovarian cancer was 100% and the positive rate for clear cell ovarian cancer was 93.8% by combining the immunological methods of β3Gal-T5 detection system and GlcNAc6ST-2 detection system. Thus; it was able to detect these types of ovarian cancer at a high rate.

### Example 7

### Measurement in Sera of Endometrial Cancer Patients by Using β3Gal-T5 Detection System

Sera of 30 cases of endometrial cancer patients and sera of 40 samples of healthy adult women, were measured using the β3Gal-T5 detection system. Among the endometrial cancer patients, 26 patients were surgically diagnosed as stage I to stage III. In one case among the 30 cases, a supervenience of cervical cancer was confirmed. In other 29 case, any cancer developments other than the endometrial cancer were not confirmed.

Step (C) of Example 4 was repeated, except that the sera of the endometrial cancer patients and healthy human diluted with PBS-T by a factor of 10 were added, instead of denatured β3Gal-T5 (E. coli). β3Gal-T5 values in the sera were determined by the standard curve (A) in FIG. 5. The results obtained from the 30 cases of the endometrial cancer patients are shown in Table 2. The value of an average+2SD in healthy adult women was defined as a cut-off point, and it was determined whether the serum of the patient was positive or negative. In this case, the average value for healthy women was 2.1 ng/mL and the cut-off point was 5.4 ng/mL. Eighteen cases (60%) among the 30 case were positive.

### Example 8

### Measurement in Sera of Endometrial Cancer Patients by Using GlcNAc6St-2 Detection System

Sera of 30 cases of the endometrial cancer patients and sera of 40 samples of the healthy adult women as tested in Example 7, were measured by using GlcNAc6ST-2 detection system.

Step (D) of Example 4 was repeated, except that the sera of the patients diluted with PBS-T by a factor of 10 were added, instead of GlcNAc6ST-2 (E. coli). GlcNAc6ST-2 values in the sera were determined by the standard curve (B) in FIG. 5. The results obtained from the 30 cases of the endometrial cancer patients were shown in Table 2. The value of an average+2SD in healthy adult women was defined as a cut-off point, and it was determined whether the sera of the patient was positive or negative. In this case, the average value for healthy women was 2.0 ng/mL and the cut-off point was 7.8 ng/mL. Ten cases (33%) among the 30 case were positive.

### Comparative Example 3

In the sera of 9 cases of the endometrial cancer patients among 30 samples as tested in Example 7, CA125 which is known as a conventional marker for endometrial cancer was measured.

The measurement of CA125 was carried out by using a kit of ECLusys reagent CA125II (Roche Diagnostics) in accordance with the attached protocol. A measurement value 35 U/mL or more was determined as positive. The results are shown in Table 2. 5 cases (38.5%) among 13 cases were positive.

### Comparative Example 4

In the sera of 19 cases of the endometrial cancer patients among the 30 cases as tested in Example 7, CA602 which is known as a conventional marker for endometrial cancer was measured.

The measurement of CA602 was carried out by using a kit of PLATE Kainos CA602 reagent (Kainos Laboratories, Inc.) in accordance with the attached protocol. A measurement value of 63 U/mL or more was determined as positive. The results are shown in Table 2. 5 cases (26.3%) among 19 cases were positive.

### Comparative Example 5

In the sera of 23 cases of the endometrial cancer patients among the 30 cases as tested in Example 7, CA19-9 which is known as a marker for pancreas cancer and a marker for endometrial cancer was measured.

The measurement of CA19-9 was carried out by using a kit of ECLusys reagent CA19-9 II (Roche Diagnostics) in accordance with the attached protocol. A measurement value of 37 U/mL or more was determined as positive. The results are shown in Table 2. 4 cases (16.7%) among 24 cases were positive.

TABLE 2

| Sample | Disease | | | | | | |
|---|---|---|---|---|---|---|---|
| number | stage | grade³⁾ | CA602 | CA125 | CA19-9 | GlcNAc6ST2 | β3Gal-T4/T5 |
| 1 | 1A | G1 | 4 | NT | 8 | 15.2 | 0 |
| 2 | 1B | G2 | 48 | NT | 22 | 262 | 0 |
| 3 | 2A | G1 | 9 | NT | 7 | 0 | 25.1 |
| 4 | 3C | G1 | NT²⁾ | 22 | 82 | 17.9 | 41.1 |
| 5 | 2A | G1 | 19 | 93 | 12 | 15.1 | 62.9 |
| 6 | 1B | G1 | 94 | NT | 88 | 0 | 49.9 |
| 7 | 3A | Carcino- | 614 | NT | 13 | 16.0 | 0 |
| | | sarcoma | | | | | |
| 8 | 1B | G1 | 6 | NT | <1 | 0 | 0 |
| 9 | 1A | G1 | 25 | NT | 24 | 0 | 0 |
| 10 | 3C | G3 | NT | 830 | 1145 | 3.0 | 0 |
| 11 | 3A | G3 | NT | 81 | 13 | 0 | 0 |
| 12 | 1B | G3 | NT | 28 | 22 | 23.6 | 21.3 |
| 13 | 1B | G1 | 21 | 26 | 19 | 0 | 0 |
| 14 | 1B | G1 | 15 | NT | NT | 9.8 | 26.4 |
| 15 | 1A | G1 | 8 | NT | 7 | 4.0 | 24.7 |
| 16 | 1B | G1 | NT | NT | NT | 6.4 | 0 |
| 17 | 1A | G1 | NT | 4 | 28 | 0 | 0 |
| 18 | 2A | G1 | 20 | NT | 28 | 3.2 | 31.8 |
| 19 | 1B | G1 | 9 | NT | 19 | 6.0 | 32.3 |
| 20 | 1C | G2 | 22 | 22 | <1 | 0 | 0 |
| 21 | 1A | G1 | 29 | NT | 7 | 5.8 | 24.7 |
| 22 | 1B | G2 | 12 | NT | 8 | 6.3 | 20.9 |
| 23 | 1C | G1 | 81 | NT | 100 | 28.6 | 29.7 |
| 24 | 2A | G1 | NT | 15 | <1 | 5.1 | 9.2 |
| 25 | 3A¹⁾ | G1 | 92 | NT | NT | 0 | 21.8 |
| 26 | 3A | G3 | 409 | NT | 15 | 0 | 0 |
| 27 | | | NT | 214 | 19 | 0 | 13.1 |
| 28 | | | NT | 84 | NT | 63.3 | 22.9 |
| 29 | | | NT | 11 | NT | 0.4 | 9.0 |
| 30 | | | NT | 25 | NT | 28.8 | 11.2 |

¹⁾complicated with cervical cancer
²⁾NT: not tested
³⁾Grade represents a degree of histodifferentiation (i.e., grade of malignancy of endometrial cancer). G1 (grade 1) indicates a well-differentiated form, and is a type which is similar to the histologically-normal gland structure. On the other hand, G3 (grade 3) indicates a poorly-differentiated form, and is a type which shows a random propagation.

### Analytic Example 2

The results shown in Table 2 were reclassified into the positive rates obtained by each of markers, as shown in Table 3.

TABLE 3
Measurement results in the sera of the endometrial cancer patients

| | Number of | Number of | Positive |
|---|---|---|---|
| | samples | positive | rate 5% |
| Antibody | (a) | samples (b) | (b/a × 100) |
| CA602 | 19 | 5 | 26.3 |
| CA125 | 13 | 5 | 38.5 |
| CA19-9 | 24 | 4 | 16.7 |
| GlcNAcST2 | 30 | 10 | 33.3 |
| β3Gal-T4/T5 | 30 | 18 | 60.0 |
| GlcNAcST2 + β3Gal-T4/T5 | 30 | 21 | 70.0 |

The positive rates for each of CA602, CA125, and CA19-9, known as conventional markers for the endometrial cancer, were 26.3%, 38.5%, and 16.7%, respectively. On the other hand, the positive rate for β3Gal-T5 detection system was 60%, and the high positive rate was confirmed. Further, the positive rate for GlcNAc6ST-2 detection system was 33.3%, which was higher than the positive rates for CA602 and CA19-9, and lower than the positive rate for CA125. However, it was revealed that the GlcNAc6ST-2 detection system can effectively detect the endometrial cancer which was not detected by the conventional markers, as shown in the sample numbers of 1, 2, and 12 of Table 2. Further, in the measurement using the β3Gal-T5 detection system and/or GlcNAc6ST-2 detection system, 70% of samples ware positive. Thus, it was possible to detect the endometrial cancer at high rates, by combining the immunological methods of the β3Gal-T5 detection system and the GlcNAc6ST-2 detection system.

### Example 9

Sera of 6 cases of the endometrial cancer patients were measured by using the β3Gal-T5 detection system. 6 cases from the present invention and 30 cases measured in Example 7 were divided into the disease stage I and disease stages II to IV. The positive rates for each of the disease stage I and disease stages II to IV are shown in Table 4.

TABLE 4
Positive rate for the β3Gal-T5 detection
system according to the stage of disease

| | Number of | Number of positive | Positive rate % |
|---|---|---|---|
| Disease stages | samples (a) | samples (b) | (b/a × 100) |
| Stage I | 21 | 12 | 57.1 |
| Stages II to IV | 15 | 11 | 73.3 |

As shown in Table 4, the positive rate for β3Gal-T5 detection system in the disease stage I of early endometrial cancer was 57.1%, and it was possible to detect the endometrial cancer at high rates, even in the early stage of disease.

### INDUSTRIAL APPLICABILITY

In accordance with the detecting method and the detecting kit for ovarian cancer according to the invention, it is possible to detect with high rates of success gynecologic cancer, particularly, ovarian cancer or endometrial cancer.

Although the present invention has been described with reference to specific embodiments, various changes and modifications obvious to those skilled in the art are possible without departing from the scope of the appended claims.

1.-14. (canceled)

15. A method for diagnosing ovarian cancer characterized in that β1,3-galactosyltransferase-5 and/or β1,3-galactosyltransferase-4 are analyzed.

16. The method according to claim 15, wherein an antibody specifically binding to β1,3-galactosyltransferase-5 and β1,3-galactosyltransferase-4 is used.

17. A method for diagnosing ovarian cancer characterized in that GlcNAc 6-O-sulfotransferase-2 in blood, serum, or plasma is analyzed.

18. The method according to claim 17, wherein an antibody specifically binding to GlcNAc 6-0-sulfotransferase-2 is used.

19. A method for diagnosing ovarian cancer characterized in that β1,3-galactosyltransferase-5 and/or β1,3-galactosyltransferase-4, and GlcNAc 6-0-sulfotransferase-2 are analyzed.

20. The method according to claim 19, wherein an antibody specifically binding to β1,3-galactosyltransferase-5 and β1,3-galactosyltransferase-4, and an antibody specifically binding to GlcNAc 6-0-sulfotransferase-2 are used.

21. A kit for diagnosing ovarian cancer characterized by containing an antibody specifically binding to β1,3-galactosyltransferase-5 and β1,3-galactosyltransferase-4 or a fragment thereof.

22. A kit for diagnosing ovarian cancer characterized in that the kit contains an antibody specifically binding to GlcNAc 6-0-sulfotransferase-2 or a fragment thereof, and GlcNAc 6-0-sulfotransferase-2 in blood, serum or plasma is analyzed.

23. A kit for diagnosing ovarian cancer characterized by containing an antibody specifically binding to β1,3-galactosyltransferase-5 and β1,3-galactosyltransferase-4 or a fragment thereof, and an antibody specifically binding to GlcNAc 6-0-sulfotransferase-2 or a fragment thereof.

24. A method for diagnosing endometrial cancer characterized in that β1,3-galactosyltransferase-5 and/or β1,3-galactosyltransferase-4 in blood, serum or plasma are analyzed.

25. The method according to claim 24, wherein an antibody specifically binding to β1,3-galactosyltransferase-5 and β1,3-galactosyltransferase-4 is used.

26. A method for diagnosing endometrial cancer characterized in that GlcNAc 6-0-sulfotransferase-2 is analyzed.

27. The method according to claim 26, wherein an antibody specifically binding to GlcNAc 6-0-sulfotransferase-2 is used.

28. A method for diagnosing endometrial cancer characterized in that β1,3-galactosyltransferase-5 and/or β1,3-galactosyltransferase-4 and GlcNAc 6-0-sulfotransferase-2 are analyzed.

29. The method according to claim 28, wherein an antibody specifically binding to β1,3-galactosyltransferase-5 and β1,3-galactosyltransferase-4 and an antibody specifically binding to GlcNAc 6-0-sulfotransferase-2 are used.

30. A kit for diagnosing endometrial cancer characterized in that the kit contains an antibody specifically binding to β1,3-galactosyltransferase-5 and β1,3-galactosyltransferase-4 or a fragment thereof, and β1,3-galactosyltransferase-5 and/or β1,3-galactosyltransferase-4 in blood, serum or plasma are analyzed.

31. A kit for diagnosing endometrial cancer characterized by containing an antibody specifically binding to GlcNAc 6-0-sulfotransferase-2 or a fragment thereof.

32. A kit for diagnosing endometrial cancer characterized by containing an antibody specifically binding to β1,3-galactosyltransferase-5 and β1,3-galactosyltransferase-4 or a fragment thereof, and an antibody specifically binding to GlcNAc 6-0-sulfotransferase-2 or a fragment thereof.
